(19) United States
(12) Patent Application Publication
(10) Pub. No.: US 2007/0086762 A1
(43) Pub. Date: Apr. 19, 2007

(54) Front end for 3D imaging camera

(75) Inventors: Michael W. O'Keefe, Cincinnati, OH (US); Jennifer L. Grace, Lakeside Park, KY (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/249,950

(22) Filed: Oct. 13, 2005

(51) Int. Cl. G03B 29/00; A61B 1/04

(52) U.S. Cl. 396/16

(57) ABSTRACT

A three-dimensional imaging system for forming a three dimensional image of an object tooth includes a projection unit capable of generating a projected light pattern and a detector unit capable of detecting an image pattern received from the object tooth. An optical transceiver unit transmits the projected pattern generated by the projection unit to the object tooth and receives the image pattern from the object tooth. The optical transceiver is formed of an integrated body having at least first and second transmitting faces and a reflecting surface that reflects light propagating within the body. In some embodiments, the projected pattern light is polarized in one polarization state and the detected image light is polarized in a second polarization state orthogonal to the first polarization state.

### FIELD OF THE INVENTION

The present invention relates to cameras that detect a three-dimensional image, and more particularly to the front end of a three-dimensional camera for dental and medical use.

### BACKGROUND

Cameras have been used in dentistry for a number of years. Initially, two-dimensional cameras were used for providing an image to the dentist and patient on a monitor. The image was used primarily for inspection purposes. More recently, three-dimensional (3D) cameras have been introduced for extracting 3D data for a tooth or of several teeth. The 3D data are used to produce a virtual 3D image of the patient's tooth or teeth. This virtual image may be used for manufacturing replacement teeth, or partial replacements, for example as might be used in a prosthesis such as a bridge or a crown. The 3D map of the patient's teeth may be also be used as a basis for making orthodontic braces. The 3D image can be used to replace the need for making a mold of the patient's mouth, which is often uncomfortable for the patient.

3D cameras project a pattern onto the target teeth, and then record the projected pattern as displayed on the target teeth: variations in the detected pattern can be correlated to the 3D shape of the target teeth. Thus, the 3D camera both projects the pattern and detects the projected pattern. Consequently, the front end of the camera, which sits in the patient's mouth, must be useful both for projecting the pattern and for receiving the pattern image from the target teeth. A conventional front end for a dental 3D camera uses either a turning prism to turn the projected image towards the teeth or a mirror on a shaft.

### SUMMARY OF THE INVENTION

The current front ends for dental 3D cameras include expensive precision optical items and, therefore, tend not to be disposable, which means that the dentist must sterilize the front end between uses with different patients. There remains a need, therefore, for an inexpensive front end to the 3D dental camera which can be disposable, but which retains the desired optical functions for the camera.

One embodiment of the present invention is directed to a three-dimensional imaging system for forming a three dimensional image of an object. The system includes a projection unit capable of generating a projected light pattern and a detector unit capable of detecting an image pattern received from the object. The system also includes an optical transceiver unit capable of transmitting the projected pattern generated by the projection unit to the object and of receiving the image pattern from the object and passing the image pattern to the detector unit. The optical transceiver is formed of an integrated body having at least first and second transmitting faces and a reflecting surface that reflects light propagating within the body.

Another embodiment of the invention is directed to a three-dimensional dental imaging system that includes a projection unit capable of generating a projected light pattern in a first polarization state and a detector unit capable of detecting an image pattern in a second polarization state orthogonal to the first polarization state, the image pattern being received from the object. The system also includes an optical transceiver unit capable of transmitting the projected pattern generated by the projection unit to the object and of receiving the image pattern from the object and passing the image pattern to the detector unit.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 schematically illustrated an exemplary embodiment of a 3D imaging system, according to principles of the present invention;

FIG. 2 schematically illustrates an exemplary embodiment of an optical transceiver unit for use in a 3D imaging system, according to principles of the present invention;

FIG. 3 schematically illustrates another exemplary embodiment of an optical transceiver unit for use in a 3D imaging system, according to principles of the present invention;

FIG. 4 schematically illustrates another exemplary embodiment of an optical transceiver unit for use in a 3D imaging system, according to principles of the present invention;

FIG. 5 schematically illustrates an exemplary embodiment of an optical transceiver unit used in a polarized 3D imaging system, according to principles of the present invention; and

FIG. 6 schematically illustrates an embodiment of a projection unit, according to principles of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is applicable to three-dimensional (3D) imaging systems, and is more particularly applicable to 3D imaging systems used in dentistry and other medical applications. The imaging system may be able to produce a 3D virtual image of a tooth or teeth in the patient's mouth. After several images have been taken, a processor may be able to “stitch together” a plurality of images so as to form a virtual 3D model of part, or all, of the patient's mouth.

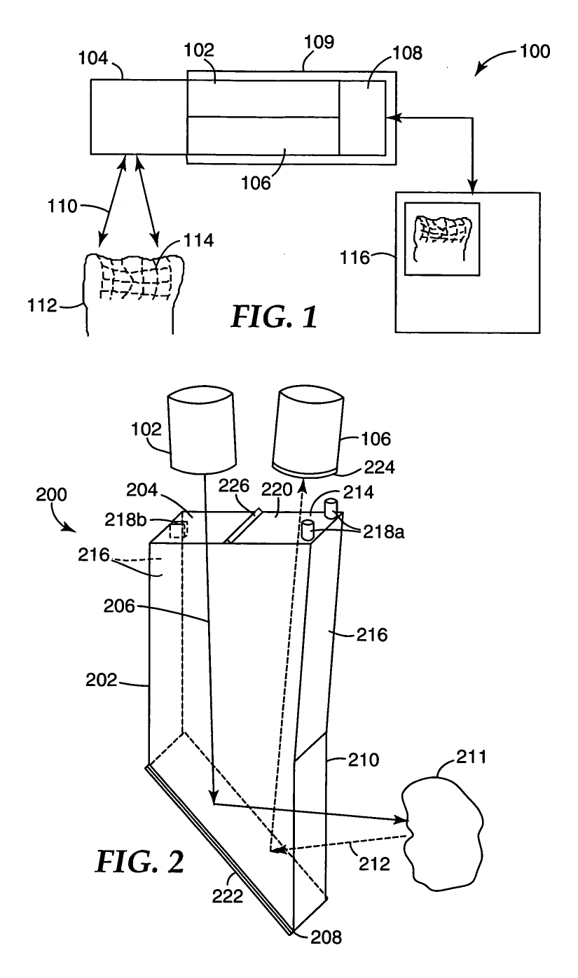

An exemplary embodiment of a 3D imaging system 100 that may be used for taking 3D images of a patient's tooth or teeth is schematically illustrated in FIG. 1. The system 100 includes a projection unit 102, an optical transceiver unit 104, a detection unit 106 and a control unit 108. The projection unit 102, detection unit 106 and control unit 108 may be housed within a housing 109. The optical transceiver unit may be wholly or partially located within the housing 109.

The projection unit 102 produces a light pattern 110 that is projected to the target 112. In the illustrated embodiment, the projected light pattern 110 is in the form of a grid pattern and the target 112 is a tooth. The projection unit 102 may use any suitable technique for producing the light pattern 110, for example the projection unit 102 may use a passive method such as a mask or series of masks, for generating the projected pattern, or may use an active method of generating a pattern, for example an actively controlled spatial light modulator. The projection unit 102 also includes projection optics for relaying the pattern to the target 112.

The optical transceiver unit 104 directs the projected pattern 110 from the projection unit 102 to the target 112, and so the target 112 is illuminated by the projected pattern 110. The pattern 114 incident on the target 112 takes on a 3D shape that is dependent on the shape of the target. The incident pattern 114 is reflected and is transmitted, via the optical transceiver unit 104 to the detection unit 106. The detection unit 106 detects the reflected pattern. The detection unit 106 may include a two dimensional photodetector array, for example a CCD array, for detecting the reflected image pattern. The detection unit 106 may also include a lens system for relaying a focused image pattern to the detector.

A control unit 108 controls the projection unit 102 and the detection unit 106. For example, the control unit 108 may control power to the projection unit 102 and, in the case of an active pattern generator, may also control the shape of the particular light pattern projected by the projection unit 102. The control unit may also perform preliminary analysis of the pattern detected by the detection unit 106. For example, where the detection unit comprises a two-dimensional detector array for detecting the reflected pattern, the control unit 108 may control the readout of the data from the two-dimensional array and may digitize the data from each detector pixel of the array.

An analyzer unit 116, which may be separate from the housing 109, may be used to form a virtual 3D image using the data from the detector array. The analyzer 116 may comprise for example, a computer or customized electronic circuitry. The information generated by the analyzer unit 116 may then be used to manufacture the desired article, for example a crown, a bridge, braces, and the like.

One exemplary embodiment of the optical transceiver unit 200 is schematically illustrated in FIG. 2. The unit 200 may be formed with a transparent body 202 using, for example a transparent polymeric or glass material. The body 202 may be formed using any suitable manufacturing technique. In some exemplary embodiments, the body may be molded, for example injection molded, using a polymeric material.

The body 202 has a first transparent face 204 that permits the projected light pattern 206 (solid line) from the projection unit 102 to pass into the body 202. It will be appreciated that only a central ray of the projected light pattern 206 is shown in the figure, to simplify the illustration. The projected light pattern 206 is reflected at a reflector 208 and is transmitted out of the body 202 through a second transparent face 210 towards the target 211. The image pattern light 212 (dashed line) reflected back from the target 211 passes back into the body 202 through the second face 210 and is directed by the reflector 208 through a third transparent face 214 to the detection unit 106.

The transparent faces 204, 210, 214 are areas where the surface of the body 202 is clear so that the image pattern light may pass through with little loss or distortion. The transparent faces 204, 210, 214 may optionally be provided with antireflection coatings to reduce reflective losses and ghosting. The other surface areas of the body, for example the sidewalls 216, may also be transparent. In some exemplary embodiments, it may be desired that the sidewalls 216 are opaque, which reduces the amount of stray light reaching the detector unit 106.

The body 202 may also be provided with alignment features for aligning the optical transceiver 200 to corresponding alignment features provided on the housing 109. The alignment features may be provided on the end 220 containing the first and third transparent faces 204, 214, or may be provided on one or more of the sidewalls 216 of the body 202. In the illustrated embodiment, the alignment features include alignment pins 218a and an alignment slot 218b provided on the end 220 containing the first and third transparent faces 204, 214. It will be appreciated that any suitable type of alignment features may be provided on the body 202 for mating to corresponding features on the housing 109 including, but not limited to, holes, slots and other types of recesses, and pins, bars and other types of protrusions.

The reflector 208 may be any suitable type of reflector. For example, the reflector may be a metallic coating, a multilayer dielectric coating or a polymer multilayer optical film (MOF) attached to the body 202. A polymer MOF includes a stack of alternating polymer layers of high and low refractive index. Polymer MOFs are discussed in greater detail in U.S. Pat. No. 6,749,427, incorporated herein by reference. The MOF reflector may be attached to the body 202 using any suitable method. For example, the MOF reflector may be attached using an adhesive or, where the body 202 is injection molded, the MOF reflector may be included as an insert in the mold prior to injecting the molten body material.

In another exemplary embodiment, the reflector 208 may reflect light within a specified wavelength range, for example the wavelength range of the light source or sources used in the projection unit 102. Thus, the reflector 208 may discriminate against ambient light, thus reducing the amount of ambient light that reaches the detection unit 106. In such a case, the outer side of the reflector 208 may optionally include an absorber 222 that absorbs the ambient light incident on the reflector 208 from outside the body 202, and that also absorbs the ambient light incident on the reflector 208 within the body 202 but which is outside the reflection bandwidth of the reflector 208.

Also, an optional filter 224 may be provided on the path of the image pattern light 212 between the target object 211 and the detector unit 106 to reduce the amount of ambient light incident at the detector unit 106. The filter 224 may be positioned, for example, between the third transparent face 214 and the detection unit 106 (as illustrated), or even between the target 211 and the second transparent face 210. The filter 224 may be, for example, an inorganic dielectric stack or a MOF filter.

Additionally, it may be desirable in some embodiments to reduce the build-up of condensation on the optical surfaces of the body 202, such as the transparent faces 204, 210, 214. In one approach, one or more of the transparent faces 204, 210, 214 may be provided with an anti-fog coating that reduces the possibility of water condensing on the faces 204, 210, 214.

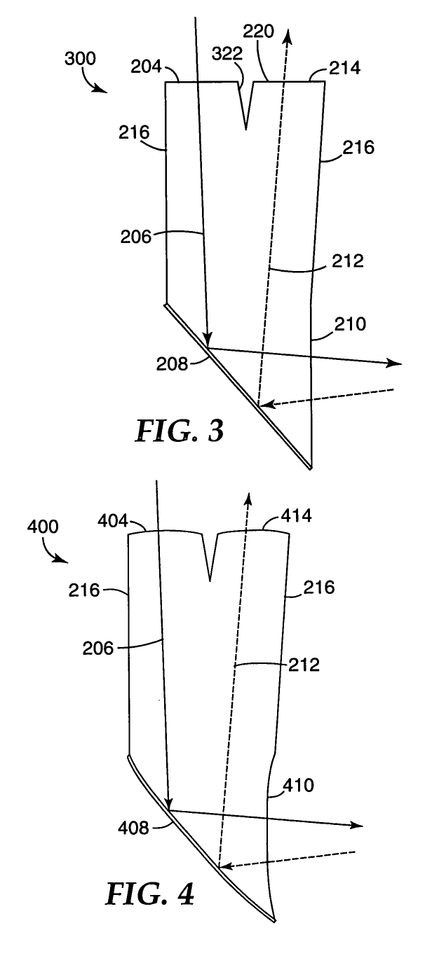

The body 202 may also include an optical path dividing element 222 that divides the optical paths of the projected light pattern 206 and the image light pattern 212. In some exemplary embodiments, the optical path dividing element 222 may be in the form of a ridge or protruding member on the end 220 facing the projection unit 102 and the detection unit 106. In other exemplary embodiments, for example the optical transceiver unit 300 schematically illustrated in FIG. 3, the optical path dividing element 322 may be in the form of a groove across the end 220.

In some embodiments of optical transceiver unit, for example the unit 200 illustrated in FIG. 2, the transparent faces 204, 210, 214 and the reflector 208 are flat. In other exemplary embodiments, for example the optical transceiver unit 400 schematically illustrated in FIG. 4, one or more of the transparent faces 404, 410, 414 may be curved. In the illustrated embodiment, all the faces 404, 410, 414 are curved, but it will be appreciated that not all the faces 404, 410, 414 need be curved.

The curved faces 404, 410, 414 provide optical power to the projected light pattern 206 and reflected image pattern 212. In addition, the reflector 408 may be curved. The projection unit 102 typically includes a projection lens system for projecting the light pattern to the object and the detection unit may include a lens system for relaying the image pattern to the detector. The curved faces 404, 410, 414 and curved reflector 408 may comprise part of these two lens systems.

The curved faces 404, 410, 414 and curved reflector 408 may have a one-dimensional curve, for example a cylindrical curve, or a two dimensional curve, for example a spherical or aspherical curve. Examples of some suitable aspherical curves include paraboloidal, ellipsoidal and toroidal curves, although the aspheric curves need not be limited to these particular profiles. A molded body 202 can easily incorporate one or more aspheric surfaces.

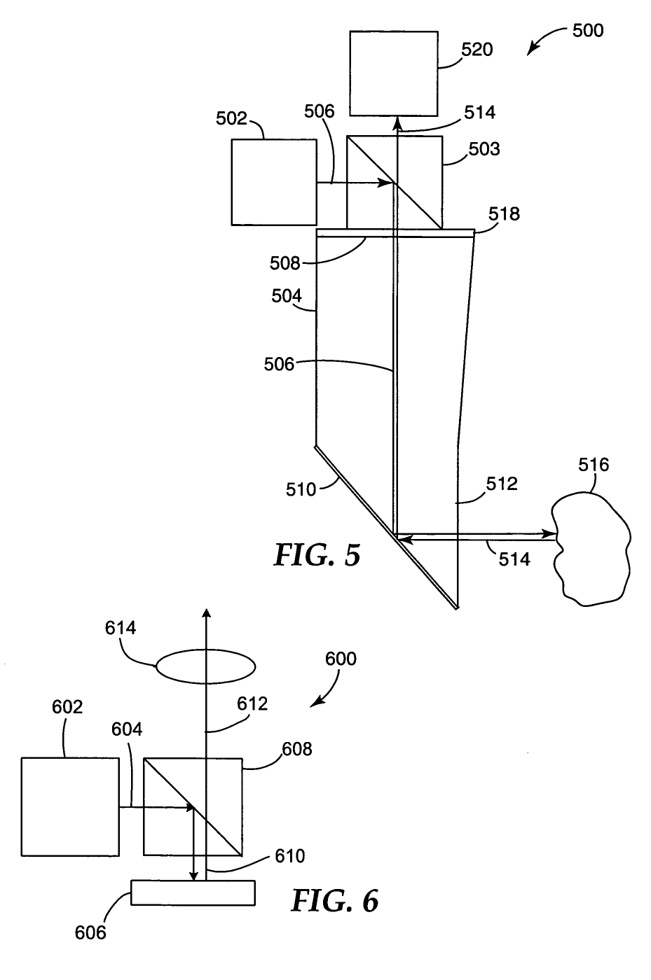

In the exemplary embodiments schematically illustrated in FIGS. 2-4, the projected light pattern and the reflected light pattern are separated within the transceiver unit following optical paths that are physically separated. Another approach to separating the projected light pattern and reflected light pattern is to use polarization separation. An exemplary embodiment of a 3D imaging system 500 that uses polarization separation for the projected and reflected image patterns is schematically illustrated in FIG. 5. The projection unit 502 produces projected light pattern 506 that is transmitted via a polarizing beamsplitter (PBS) 503 to the optical transceiver unit 504. The projected pattern light 506 passes through a first transparent face 508 into the body of the optical transceiver unit 504 and is reflected at a reflector 510 out of the body through a second transparent face 512 to the target 516. The image pattern light 514, reflected from the target 516, passes backwards along substantially the same path as the projected pattern light 506 within the optical transceiver unit 504. In the illustration, the paths of the projected pattern light 506 and the image pattern light 514 are shown slightly separated from each other, for illustrative purposes only.

At some point between the PBS 503 and the target 516, the light passes through a polarization converter 518, for example a quarter-wave retardation element. In the illustrated embodiment, the polarization converter 518 is located between the PBS 503 and the optical transceiver unit 504. In other embodiments, the polarization converter 518 may be placed within the optical transceiver unit 504, for example it may be integrated between the body of the optical transceiver unit 504 and the reflector 510, or may be placed at the second transparent face 512. Thus, at least some of the image pattern light 514 reaches the PBS 503 in a polarization state that is orthogonal to the polarization state of the projected pattern light 506 that leaves the PBS 503. Thus, the image pattern light 514 passes along a different path within the PBS 503 from the projected pattern light 506, and is directed to the detection unit 520. For example, where the PBS 503 reflects the projected pattern light 506, the image pattern light 514 may be transmitted through the PBS 503. Likewise, where the projected pattern light 506 is transmitted through the PBS 503, the image pattern light 514 may be reflected by the PBS 503.

An optical transceiver unit that is formed from a single, integrated piece as described above has potential advantages over conventional approaches that use multiple pieces. The single integrated piece may be molded, so may be replicated relatively inexpensively with a good degree of precision. This eliminates the need for the assembly, alignment and control of multiple components. Furthermore, the unit may become a disposable item. The alignment of the optical transceiver unit to the rest of the imaging system may be easily achieved using alignment features molded with the piece. Furthermore, the solid body provides optical rigidity and does not readily deform when pressed against a part of the patient, for example the patient's mouth, teeth or gums. The solid body serves to block other objects or parts of the anatomy, such as the patient's tongue, from entering the optical path while the 3D measurements as being made. Also, the solid body does not require extra wall thickness outside of the optical path, since the body itself provides the structure. The size of the optical transceiver unit, therefore is smaller than other designs that require support structure outside of the optical path, which is an important consideration for intraoral devices and other devices that have to access a restricted volume.

One exemplary embodiment of a projection unit 600 that generates a polarized light pattern for projection is schematically illustrated in FIG. 6. A light source 602 directs light 604 to a liquid crystal display (LCD) panel 606. The light source 602 may be any suitable type of light source, such as a lamp, one or more light emitting diodes, a laser, or the like. The light source 602 may generate unpolarized light or polarized light. The LCD panel 606 may be reflective, as shown, or transmissive. In this embodiment, the light 604 is directed to the LCD panel 606 via a reflective polarizer 608. The reflective polarizer 608 may be any suitable type of reflective polarizer, such as a wire grid polarizer, a multilayer optical film polarizer, or the like. The reflective polarizer 608 may be in the form of a film or may be part of a polarizing beamsplitter cube, as illustrated.

The LCD panel 606 polarization modulates some of the light 604 thereon. The modulated light 610 passes back through the polarizer 606 and is emitted as a polarized light pattern 612 that is then projected via a projection lens system 614 to the object tooth.

It will be appreciated that other arrangements may be used for generating a polarized light pattern. For example, the light source may illuminate the LCD panel using light that is transmitted through the polarizer, with the polarization modulated light being reflected by the polarizer. The use of an LCD panel permits the image projected on the object tooth to be dynamic, which may benefit the signal processing of the detected signal. This is not necessary, however, and a static image may be formed, for example by illuminating a pattern mask with polarized light.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

We claim:

1. A three-dimensional imaging system for forming a three dimensional image of an object, comprising:
a projection unit capable of generating a projected light pattern;
a detector unit capable of detecting an image pattern received from the object; and
an optical transceiver unit capable of transmitting the projected pattern generated by the projection unit to the object and of receiving the image pattern from the object and passing the image pattern to the detector unit, the optical transceiver being formed of an integrated body having at least first and second transmitting faces and a reflecting surface that reflects light propagating within the body.

2. A system as recited in claim 1, wherein the projected pattern generated by the projection unit contains polarized light.

3. A system as recited in claim 2, further comprising a polarizing beamsplitter, the projected pattern passing into the first transmitting face from the projection unit via the polarizing beamsplitter along a first optical path and the image pattern passing from the first transmitting face to the detector unit via the polarizing beamsplitter along a second optical path different from the first optical path.

4. A system as recited in claim 1, wherein the body is formed of a solid, substantially transparent material.

5. A system as recited in claim 1, wherein the projected pattern generated by the projection unit contains unpolarized light.

6. A system as recited in claim 1, wherein the projected pattern passes into the body via the first transmitting face, the projected pattern passes out of the body to the object via the second transmitting face and the image pattern passes into the body from the object via the second transmitting face.

7. A system as recited in claim 6, the body further comprising a third transmitting face, the image pattern passing out of the body through the third transmitting face to the detector unit.

8. A system as recited in claim 1, wherein at least one of the first and second transmitting face provides optical power to light passing therethrough.

9. A system as recited in claim 1, wherein the body comprises a third transmitting face, the image pattern being transmitted out the body through the third face to the detection unit, the third transmitting face being curved.

10. A system as recited in claim 1, wherein the reflecting surface is curved.

11. A system as recited in claim 1, wherein the reflecting surface is flat.

12. A system as recited in claim 1, wherein the reflecting surface comprises a metalized mirror surface.

13. A system as recited in claim 1, wherein the reflecting surface comprises a multilayer optical film.

14. A system as recited in claim 1, wherein the body has opaque sidewalls.

15. A system as recited in claim 1, wherein the body further comprises an optical path dividing element to divide a first optical path for the projected pattern and a second optical path for the image pattern.

16. A system as recited in claim 15, wherein the optical path dividing element comprises an member protruding from the surface of the body.

17. A system as recited in claim 15, wherein the optical path dividing element comprises a recess in the body.

18. A system as recited in claim 1, wherein the system further comprises a housing and the body further comprises integrated alignment features for aligning the body to the housing.

19. A system as recited in claim 1, further comprising a control unit coupled to control operation of the projection unit and the detection unit, and an analyzer unit coupled to the control unit that produces a virtual 3D image of the target using data received from the detection unit.

20. A three-dimensional dental imaging system, comprising:
a projection unit capable of generating a projected light pattern in a first polarization state;
a detector unit capable of detecting an image pattern in a second polarization state orthogonal to the first polarization state, the image pattern being received from the object ; and
an optical transceiver unit capable of transmitting the projected pattern generated by the projection unit to the object and of receiving the image pattern from the object and passing the image pattern to the detector unit.

21. A system as recited in claim 20, further comprising a polarizing beamsplitter, the projected pattern passing into the first transmitting face from the projection unit via the polarizing beamsplitter along a first optical path and the image pattern passing from the first transmitting face to the detector unit via the polarizing beamsplitter along a second optical path different from the first optical path.

22. A system as recited in claim 20, wherein the projection unit comprises a liquid crystal display panel for generating the polarized pattern light.

23. A system as recited in claim 20, wherein the optical transceiver is formed of an integrated body having at least first and second transmitting faces and a reflecting surface that reflects light propagating within the body.

24. A system as recited in claim 23, wherein the body is formed of a solid, substantially transparent material.

25. A system as recited in claim 23, wherein the projected pattern passes into the body via the first transmitting face, the projected pattern passes out of the body to the object via the second transmitting face and the image pattern passes into the body from the object via the second transmitting face.

26. A system as recited in claim 25, the body further comprising a third transmitting face, the image pattern passing out of the body through the third transmitting face to the detector unit.

27. A system as recited in claim 23, wherein at least one of the first and second transmitting faces provides optical power to light passing therethrough.

28. A system as recited in claim 23, wherein the body comprises a third transmitting face, the image pattern being transmitted out the body through the third face to the detection unit, the third transmitting face being curved.

29. A system as recited in claim 23, wherein the reflecting surface is curved.

30. A system as recited in claim 23, wherein the reflecting surface is flat.

31. A system as recited in claim 23, wherein the body comprises opaque sidewalls.

32. A system as recited in claim 23, wherein the system further comprises a housing and the body further comprises integrated alignment features for aligning the body to the housing.

33. A system as recited in claim 20, further comprising a control unit coupled to control operation of the projection unit and the detection unit, and an analyzer unit coupled to the control unit that produces a virtual 3D image of the target using data received from the detection unit.
